(19) United States
(12) Patent Application Publication
(10) Pub. No.: US 2023/0040999 A1
(43) Pub. Date: Feb. 9, 2023

(54) SYSTEMS AND METHODS FOR FACILITATING MANAGING OF MEDICAL RECORDS

(71) Applicant: Suzanne Webb, Glendale, AZ (US)

(72) Inventor: Suzanne Webb, Glendale, AZ (US)

(21) Appl. No.: 17/881,056

(22) Filed: Aug. 4, 2022

Related U.S. Application Data
(60) Provisional application No. 63/229,426, filed on Aug. 4, 2021.

(51) Int. Cl. G16H 10/60; G16H 40/67

(52) U.S. Cl. CPC G16H 10/60; G16H 40/67; G16H 50/20

(57) ABSTRACT

The present invention provides a system and method for facilitating managing of medical records. The system and method comprise steps of receiving medical data from a user device, analyzing the medical data, receiving a categorizing keyword from the user device, analyzing the categorizing keyword to identify a category type, organizing the medical records based on the category type, storing the medical data in a database, transmitting organized medical records to a medical expert's device, receiving expert data from the medical expert's device; and storing the expert data with the medical data in the database.

### FIELD OF THE INVENTION

The present invention relates generally to a field of data processing. More specifically, the present invention comprises systems, methods, and applications for facilitating and managing of medical records.

### BACKGROUND OF THE INVENTION

The field of data processing is technologically important to several industries, business organizations and/or individuals. In particular, the use of medical records is prevalent for tracking and recording various medical treatment acquired by the patient.

With regard to a patient's medical treatment, his/her medical reports, treatment data, diagnostics results etc. are all controlled and managed by patients utilizing hospital records and authorities. In this way, the hospital "novel" authorities own these medical data despite the fact that real ownership of the medical data resides with the patient itself. This makes it difficult for the patient to correlate with medical data of some other treatment done from another hospital or a medical professional. Conventionally, the process of storing records is done through an Electronic Health Record (EHR) software. However, the topology of medical interactions stem from a pre-specified healthcare authority with little to no interoperability for the patient to derive meaningful insights from said interactions. Essentially the patient is unable to record, track and maintain the medical data according to his convenience and he has no where to store his records. Further, there is an added risk of security and trust unless the medical data are maintained by a secure software platform.

Existing techniques for managing medical records are deficient with regard to several aspects, because they are scattered amongst various providers, surgeons, labs, records of various natures which don't communicate as one and are siloed by EMRs. For instance, current medical records management applications are unable to determine health conditions of a user using various medical data, test results, etc. provided by the user. Subsequently, current technologies are unable to determine diseases and illnesses that may or may not be acquired by the user by analyzing past vaccination records. Furthermore, current technologies are not able to acquire and maintain records of test results, readings and measurements associated with various medical devices. Furthermore, current technologies are not able to effectively analyze data from medical devices to predict patient's health and include them as daily medical reports. Moreover, current technologies do not determine an effective treatment plausible for the user using his/her medical records and accordingly schedule an appointment with a medical provider.

Therefore, there is a need for improved methods and systems to facilitate managing of medical records that may overcome one or more of the above-mentioned problems and/or limitations.

### SUMMARY

This summary is provided to introduce a selection of concepts in a simplified form, which are further described below in the Detailed Description. This summary is not intended to be used to limit the claimed subject matter's scope..

According to some embodiments, a system for facilitating managing of medical records is disclosed. Accordingly, the system may include a communication device, a processing device, and a storage device. Further, the communication device may be configured for receiving medical data from a user device. Further, the communication device may be configured for receiving a categorizing keyword from the user device. Additionally,, the communication device may be configured for transmitting organized medical records to a medical expert's device. Further, the communication device may be configured for receiving expert data from the medical expert's device. Further, the processing device may be communicatively coupled or internalized with the communication device. Further, the processing device may be configured for analyzing the medical data. Further, the processing device may be configured for analyzing the categorizing keyword to identify a category type. Further, the processing device may be configured for organizing medical records based on the category type. Further, the storage device may be communicatively coupled with the processing device. Further, the storage device may be configured for storing the medical data in a database. Further, the storage device may be configured for storing the expert data with the medical data in the database.

According to some aspects, a method for facilitating digital voting is disclosed. Accordingly, the method may include a step of receiving, using a communication device, medical data from a user device. Further, the method may include a step of analyzing, using a processing device, the medical data based on the receiving. Further, the method may include a step of receiving, using the communication device, a categorizing keyword from the user device. Further, the method may include a step of analyzing, using the processing device, the categorizing keyword to identify a category type. Further, the method may include a step of organizing, using the processing device, the medical records based on the category type. Further, the method may include a step of storing, using a storage device, the medical data in a database. Further, the method may include a step of transmitting, using the communication device, organized medical records to a medical expert's device. Further, the method may include a step of receiving, using the communication device, expert data from the medical expert's device. Further, the method may include a step of storing, using the storage device, the expert data with the medical data in the database.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. While the present teachings will be described in conjunction with various embodiments, it will be understood that they are not intended to limit the present teachings to those embodiments. On the contrary, the present teachings are intended to cover various alternatives, modifications, and equivalents, as will be appreciated by those skilled in the art.

FIG. 1 is an illustration of an online platform consistent with various embodiments of the present disclosure.

FIG. 2 is a block diagram of a system for facilitating managing of medical records, in accordance with some embodiments.

FIG. 3 is a flowchart for facilitating managing of medical records by a user, in accordance with some embodiments.

FIG. 4 is a flowchart for facilitating examining of medical records associated with a user, in accordance with some embodiments.

FIG. 5 is a flowchart for facilitating managing of data from medical devices, in accordance with some embodiments.

FIG. 6 is a flowchart for facilitating scheduling appointments using medical records, in accordance with some embodiments.

FIG. 7 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being "preferred" is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, "a" and "an" each generally denotes "at least one," but does not exclude a plurality unless the contextual use dictates otherwise. When used herein to join a list of items, "or" denotes "at least one of the items," but does not exclude a plurality of items of the list. Finally, when used herein to join a list of items, "and" denotes "all of the items of the list."

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of systems and methods for facilitating managing of medical records, embodiments of the present disclosure are not limited to use only in this context.

### Overview

The present disclosure describes systems and methods for facilitating managing of medical records.

In an instance, the present disclosure describes ES an Digital Health Record app. Further, the ES Digital Health Record app may contain medical records for individuals. Rather being owned by a medical organization the medical records in the ES Digital Health Record app are owned by the respective individuals. Further, the ES Digital Health Record app may contain details like date of birth, current picture, height, weight, blood pressure, DNA report, vaccination records, surgery records, medical conditions and so forth. The ES Digital Health Record app may include information like prescriptions and doctors' orders on how to take medications, genetic testing and compliance with prescriptions and over the counter medications. Further, the ES Digital Health Record app may track diet pattern, reaction to medications, activity level to maintain overall health. Further, all the records in the ES Digital Health Record app would be maintained by patients, and they would have the ability to share the data with their medical providers.

Further, the present disclosure describes a technology stack implemented by the ESS Digital Health Record app. Further, the technology stack may include backend technologies, front end (mobile application), additional libraries, etc. The architectural technologies may include Application Programming interfaces (REST) Flutter for mobile development, Linux instances for operating system, Apache server, and Kubernetes for replication, relational databases, . The backend technologies may include SQL as database and Amazon S3 as storage. Further, the backend technologies also include GraphQL and Github code repository. Further, the front end (mobile application) may include React Native, React Navigation, Redux, Axios, Pritter, etc. Further, the additional libraries may include PDF reader, FaceID, Touch authentication, passcode authentication, Background Uploader v7, tag adding concept, Notification plugin, CL/CD for app builds, etc.

Further, the present disclosure describes an architecture for ES Digital Health. The ES Digital Health is a patient data management application. Further, the ES Digital Health may use AWS (Amazon Web Services) to host its platform, thereby taking advantage of an inherent security that AWS offers. Subsequently, the architecture may include a frontend mobile app and a backend server. The frontend mobile app may implement the React native framework and Typescript language. Further, the ES Digital Health may implement services like AWS Amplify, AWS Cognito, AWS AppSync, Amazon DynamoDB, Amazon S3, Lambda, JavaScript, etc. for the backend server. Further, the AWS Amplify may be used for authentication of a user. After authentication, the AWS Cognito may manage the sign-up and sign-in functionalities of the ES Digital Health application. The AWS AppSync service may manage database queries using GraphQL language. The Amazon DynamoDB may be a database to store user information of all the users. The Amazon S3 may be used as a storage service for storing files, codes, etc.

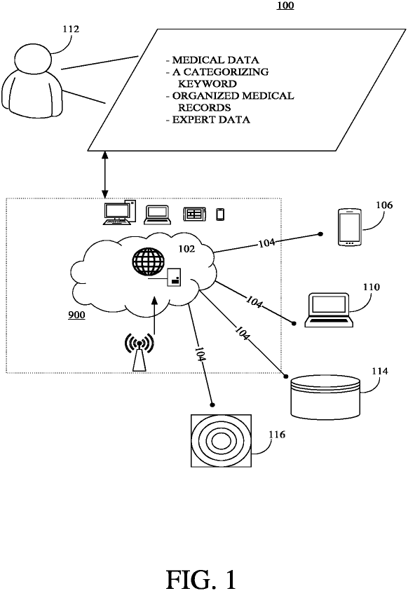

FIG. 1 is an illustration of an online platform 100 consistent with various embodiments of the present disclosure. By way of non-limiting example, the online platform 100 for facilitating managing of medical records may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, sensors 116, actuators (not shown) and an apparatus 118 over a communication network 104, such as, but not limited to, the Internet. Further, users of the online platform 100 may include relevant parties such as, but not limited to, end-users and administrators. Accordingly, in some instances, electronic devices operated by the one or more relevant parties may be in communication with the online platform 100.

A user 112, such as the one or more relevant parties, may access the online platform 100 through a web-based software application or browser. The web-based software application may be embodied as, for example, but not be limited to, a website, a web application, a desktop application, and a mobile application compatible with a computing device 900.

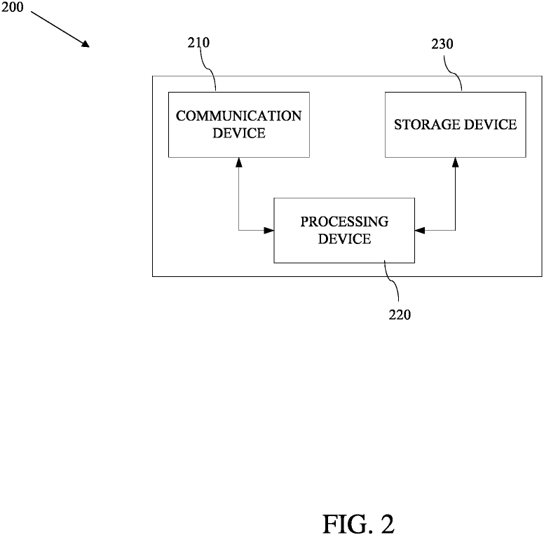

FIG. 2 is a block diagram of a system for facilitating managing of medical records 200, in accordance with some embodiments. Accordingly, the system 200 may include a communication device 210, a processing device 220, and a storage device 230.

Further, the communication device 210 may be configured for receiving medical data from a user device. Further, the user device may be associated with a user. Further, the user may be a patient of a medical organization, a medical professional, etc. Further, the medical data may include user information like name, age, gender, height weight, etc. of the user. Further, the medical data may include medical records of past and ongoing treatments of the user. Further, the medical records may include vaccination records, surgery records, prescriptions, medications, medical conditions, medical test results, lab results and genetic testing, diagnosis results and so on. Further, the medical data may include contextual information like date, time, location, venue, examiner, medical organization, medical professional, etc. associated with treatment. Subsequently, the medical data may be in image, text, video, audio and other data formats. Further, the communication device 210 may be configured for receiving a categorizing keyword from the user device. Further, the communication device 210 may be configured for transmitting organized medical records to a medical expert's device. Further, the medical expert's device may be associated with a medical expert. Further, the medical expert may examine and study the organized medical records for the treatment of an illness. Further, the communication device 210 may be configured for receiving expert data from the medical expert's device. Further, the expert data may include recommendations, prescriptions, treatment, diet plans, etc. for the illness. Further, the expert data may be associated with the medical data of the user.

Further, the processing device 220 may be communicatively coupled with the communication device 210. Further, the processing device 220 may be configured for analyzing the medical data based on the receiving. Further, the processing device 220 may be configured for analyzing the categorizing keyword to identify a category type. Further, the category type may include a treatment type, a surgery type, a prescription type, a treatment period type, a medical condition type, a medical organization type, a medical professional type, etc. Further, the processing device 220 may be configured for organizing the medical records based on the category type. Further, the treatment type category may include organizing the medical records based on a treatment undergone by the user. Further, the surgery type may include organizing the medical records based on a surgery undergone by the user. Further, the prescription type may include organizing the medical records based on prescriptions assigned to the user. Further, the treatment period type may include organizing the medical records based on duration of treatment undergone by the user. Further, the medical condition type may include organizing the medical records associated with a medical condition of the user. Further, the medical organization type may include organizing the medical records associated with a medical organization treating the user. Similarly, the medical professional type may include organizing the medical records associated with a medical professional treating the user.

Further, the storage device 230 may be communicatively coupled with the processing device. Further, the storage device 230 may be configured for storing the medical data in a database. Further, the medical data may include the organized medical records. Further, the storage device 230 may be configured for storing the expert data with the medical data in the database.

Further, in some embodiments, the communication device 210 may be configured for transmitting a safety report to the user device associated with the user. Further, the processing device 220 may be configured for analyzing the expert data and the medical data. Further, the processing device 220 may be configured for determining susceptibility and treatment of illnesses based on the analyzing. Further, the processing device 220 may be configured for generating the safety report based on the determining. Further, the safety report may include a list of illnesses. Further, the list of illnesses may include infections, allergies, diseases, etc. that may be acquired by the user. Further, the safety report may include a list of medications, remedies, etc. for the list of illnesses. Further, the storage device 230 may be configured for retrieving the expert data and the medical data associated with a user. Further, the expert data may include recommendations, prescriptions, treatments, diet plans, etc. for an illness. Further, the medical data may include vaccination records, surgery records, prescriptions, medications, medical conditions, medical test results, diagnosis results, etc.

Further, in some embodiments, the communication device 210 may be configured for receiving a pairing key from at least one medical device. Further, the at least one medical device may be associated with the user. Further, the at least one medical device may include medical equipment like Blood Pressure monitor, Blood Glucose monitor, ECG machine, EEG machine, pacemakers, activity trackers, etc. Further, the at least one medical device may contain a wireless module to exchange data wirelessly. Further, the wireless module may include a Bluetooth module, a Wi-Fi module, etc. Further, the communication device 210 may be configured for receiving device data from the at least one medical device. Further, the device data may include readings, test results, graphs, etc. from the at least one medical device. Further, the processing device 220 may be configured for analyzing the pairing key based on the receiving. Further, a software platform may register the at least one medical device with user information associated with the user using the pairing key. Further, the processing device 220 may be configured for analyzing the device data based on the receiving. Further, the analyzing of the device data may detect abnormalities in health conditions. Further, the abnormalities in health conditions may arise if the device data is out of a predefined range. Further, the predefined range may be set by a medical professional. Further, the processing device 220 may be configured for generating a health report based on analyzing the device data. Further, the health report may be associated with date, time, type, etc. of a medical test. Further, the health report may convey present health conditions and possible health risks associated with the user. Further, the storage device 230 may be configured for storing the health report in a database based on the generating. Further, the health report may be coupled with the medical data of the user.

Further, in some embodiments, the communication device 210 may be configured for receiving a schedule request from the user device. Further, the schedule request may include user information like name, age, gender, height weight, etc. of the user. Further, the communication device 210 may be configured for transmitting at least one appointment preset to the user device. Further, the communication device 210 may be configured for receiving an appointment preset selection from the at least one appointment preset from the user device. Further, the appointment preset selection may include selection of a data, time, venue, etc. for the at least one medical treatment associated with the appointment. Further, the communication device 210 may be configured for transmitting an alert based on at least one schedule reminder. Further, the alert may be associated with a notification that may include a textual message and an alert sound. Further, the processing device 220 may be configured for analyzing the schedule request to identify the medical data associated with the user. Further, the processing device 220 may be configured for determining a treatment type using the medical data. Further, the treatment type may be associated with at least one medical treatment. Further, the at least one medical treatment may include a medical diagnosis, surgery, a medical test, a vaccination drive, etc. Further, the processing device 220 may be configured for generating the at least one schedule reminder based on the appointment preset selection. Further, the storage device 230 may be configured for retrieving the medical data based on the analyzing. Further, the storage device 230 may be configured for retrieving the at least one appointment preset associated with the treatment type. Further, the at least one appointment preset may include dates, times, venues, etc. for an appointment associated with the at least one medical treatment.

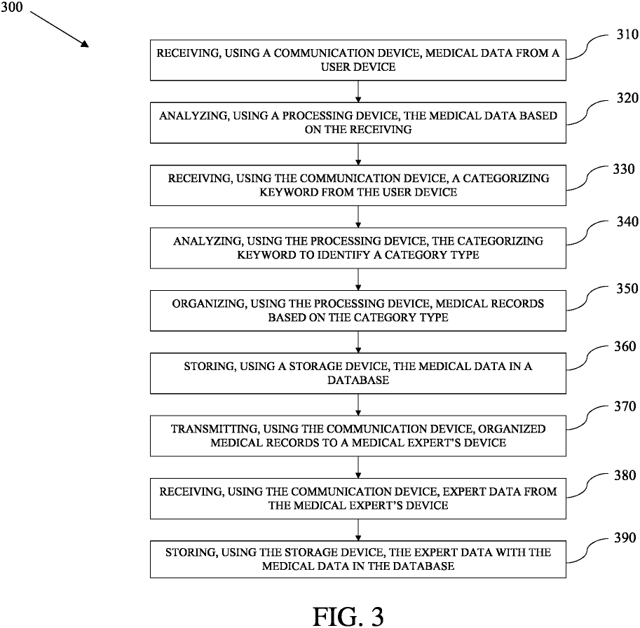

FIG. 3 is a flowchart for facilitating managing of medical records by a user, in accordance with some embodiments. Accordingly, the managing method 300 may include a step of receiving, using a communication device 210, medical data from a user device, at 310. Further, the user device may be associated with the user. Further, the user may be a patient of a medical organization, a medical professional, etc. Further, the medical data may include user information like name, age, gender, height weight, etc. of the user. Further, the medical data may include medical records of past and ongoing treatments of the user. Further, the medical records may include vaccination records, surgery records, prescriptions, medications, medical conditions, medical test results, diagnosis results and so on. Further, the medical data may include information like date, time, location, venue, examiner, medical organization, medical professional, etc. associated with treatment. Subsequently, the medical data may be in image, text, video, audio and other data formats. The medical data can include PDF, JPG, JPEG, DICOM, spreadsheet formats.

Further, the method 300 may include a step of analyzing, using a processing device 220 to obtain the health of the patient and changes that could result in better health, the medical data based on the receiving, at 320.

Further, the method 300 may include a step of receiving, using the communication device 210, a categorizing keyword from the user device, at 320.

Further, the method 300 may include a step of analyzing, using the processing device 220, the categorizing keyword to identify a category type, at 330. Further, the category type may include a treatment type, a surgery type, a prescription type, a treatment period type, a medical condition type, a medical organization type, a medical professional type, etc.

Further, at 350, the method 300 may include a step of organizing, using the processing device 220, the medical records based on the category type. Further, the treatment type category may include organizing the medical records based on a treatment undergone by the user. Further, the surgery type may include organizing the medical records based on a surgery undergone by the user. Further, the prescription type may include organizing the medical records based on prescriptions assigned to the user. Further, the treatment period type may include organizing the medical records based on duration of treatment undergone by the user. Further, the medical condition type may include organizing the medical records associated with a medical condition of the user. Further, the medical organization type may include organizing the medical records associated with a medical organization treating the user. Similarly, the medical professional type may include organizing the medical records associated with a medical professional treating the user.

Further, the method 300 may include a step of storing, using a storage device 230, the medical data in a database, at 360. Further, the medical data may include organized medical records.

Further, the method 300 may include a step of transmitting, using the communication device 210, the organized medical records to a medical expert's device, at 370. Further, the medical expert's device may be associated with a medical expert. Further, the medical expert may examine and study the organized medical records for the treatment of an illness. Accordingly, the medical expert may advise recommendations, prescriptions, treatments, diet plans, etc. for the user.

Further, the method 300 may include a step of receiving, using the communication device 210, expert data from the medical expert's device, at 380. Further, the expert data may include recommendations, prescriptions, treatment, diet plans, etc. for the illness. Further, the expert data may be associated with the medical data of the user.

Further, the method 300 may include a step of storing, using the storage device, 230, the expert data with the medical data in the database, at 390.

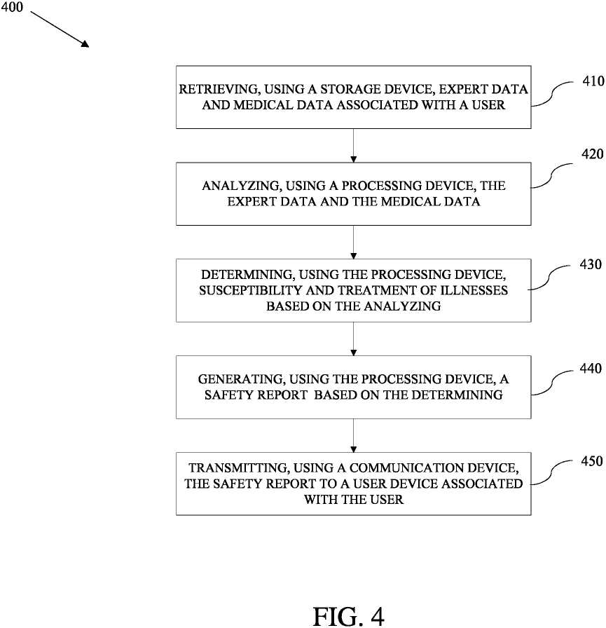

FIG. 4 is a flowchart for facilitating examining of medical records associated with a user, in accordance with some embodiments. Accordingly, the examining method 400 may include retrieving, using a storage device 230, expert data and medical data associated with the user, at 410. Further, the expert data may include recommendations, prescriptions, treatments, diet plans, etc. for an illness. Further, the medical data may include vaccination records, surgery records, prescriptions, medications, medical conditions, medical test results, diagnosis results, etc.

Further, the method 400 may include a step of analyzing, using a processing device 220, the expert data and the medical data, at 420.

Further, the method 400 may include a step of determining, using the processing device 220, susceptibility and treatment of illnesses based on the analyzing, at 430. Further, the susceptibility of the illnesses may be determined using past history of diseases, medical conditions, medical test results, ongoing treatments, vaccines acquired, etc. from the medical data. Further, the treatment of the illnesses may be determined using recommendations, prescriptions, medications, diet plans, etc. using the expert data.

Further, the method 400 may include a step of generating, using the processing device 220, a safety report based on the determining, at 440. Further, the safety report may include a list of illnesses. Further, the list of illnesses may include infections, allergies, diseases, etc. that may be acquired by the user. Further, the safety report may include a list of medications, remedies, etc. for the list of illnesses.

Further, the method 400 may include a step of transmitting, using a communication device 210, the safety report to a user device associated with the user, at 450.

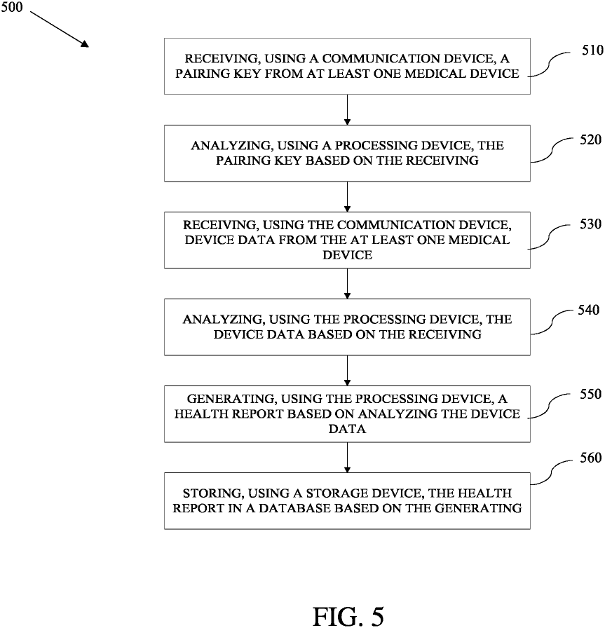

FIG. 5 is a flowchart for facilitating managing of data from medical devices, in accordance with some embodiments. Accordingly, the data managing method 500 may include a step of receiving, using a communication device 210, a pairing key from at least one medical device, at 510. Further, the at least one medical device may be associated with a user. Further, the at least one medical device may include medical equipment like Blood Pressure monitor, Blood Glucose monitor, ECG machine, EEG machine, pacemakers, activity trackers, etc. Further, the at least one medical device may contain a wireless module to exchange data wirelessly. Further, the wireless module may include a Bluetooth module, a Wi-Fi module, etc.

Further, the method 500 may include a step of analyzing, using a processing device 220, the pairing key based on the receiving, at 520. Further, a software platform may register the at least one medical device with user information associated with the user using the pairing key.

Further, the method 500 may include a step of receiving, using the communication device 210, device data from the at least one medical device at 530. Further, the device data may include readings, test results, graphs, etc. from the at least one medical device.

Further, the method 500 may include a step of analyzing, using the processing device 220 for the purpose of providing information to the patient for the purpose of better health, the device data based on the receiving, at 540. Further, the analyzing of the device data may detect abnormalities in health conditions. Further, the abnormalities in health conditions may arise if the device data is out of a predefined range. Further, the predefined range may be set by a medical professional.

Further, the method 500 may include a step of generating, using the processing device 220, a health report based on analyzing the device data, at 550. Further, the health report may be associated with date, time, type, etc. of a medical test. Further, the health report may convey present health conditions and possible health risks associated with the user.

Further, the method 500 may include a step of storing, using a storage device 230, the health report in a database based on the generating, at 560. Further, the health report may be coupled with the medical data of the user.

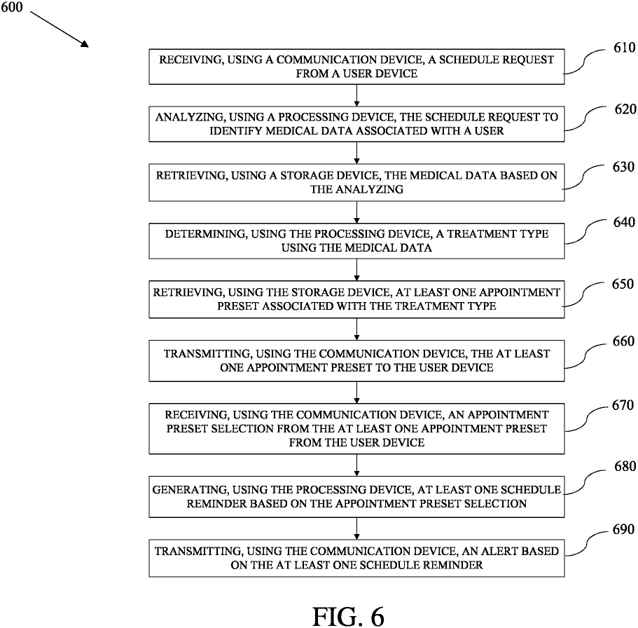

FIG. 6 is a flowchart for facilitating scheduling appointments using medical records, in accordance with some embodiments. Accordingly, the scheduling method 600 may include a step of receiving, using a communication device 210, a schedule request from a user device, at 610. Further, the user device may be associated with a user. Further, the schedule request may include user information like name, age, gender, height weight, etc. of the user.

Further, the method 600 may include a step of analyzing, using a processing device 220, the schedule request to identify medical data associated with the user, at 620.

Further, the method 600 may include a step of retrieving, using a storage device 230, the medical data based on the analyzing, at 630.

Further, the method 600 may include a step of determining, using the processing device 220, a treatment type using the medical data, at 640. Further, the treatment type may be associated with at least one medical treatment. Further, the at least one medical treatment may include a medical diagnosis, surgery, a medical test, a vaccination drive, etc. Further, the at least one medical treatment may be performed by a medical professional.

Further, the method 600 may include a step of retrieving, using the storage device 230, at least one appointment preset associated with the treatment type, at 650. Further, the at least one appointment preset may include dates, times, venues, etc. for an appointment associated with the at least one medical treatment.

Further, the method 600 may include a step of transmitting, using the communication device 210, the at least one appointment preset to the user device, at 660.

Further, the method 600 may include a step of receiving, using the communication device 210, an appointment preset selection from the at least one appointment preset from the user device, at 670. Further, the appointment preset selection may include selection of a data, time, venue, etc. for the at least one medical treatment associated with the appointment.

Further, the method 600 may include a step of generating, using the processing device 220, at least one schedule reminder based on the appointment preset selection, at 680. Further, the at least one schedule reminder may assist the user to carry out the appointment.

Further, the method 600 may include a step of transmitting, using the communication device 210, an alert based on the at least one schedule reminder, at 690. Further, the alert may be associated with a notification that may include a textual message and an alert sound. Further, the alert may provide the details associated with the appointment preset selection and notify the user to complete the appointment.

In some embodiments, the present invention may include ES Digital Health application. Further, the ES Digital Health application is a patient data management application. Further, the ES Digital Health may use AWS (Amazon Web Services) to host the software platform, thereby taking advantage of an inherent security that AWS offers. Further, the architecture may include a frontend mobile app and a backend server. Further, the frontend mobile app may implement React native framework and Typescript language. Further, the ES Digital Health may implement services like AWS Amplify, AWS Cognito, AWS AppSync, Amazon DynamoDB, Amazon S3, Lambda, JavaScript, etc. for the backend server. Further, the AWS Amplify may be used for authentication of a user. After authentication, the AWS Cognito may manage the sign-up and sign-in functionalities of the ES Digital Health application. Further, the AWS AppSync service may manage database queries using GraphQL language. Further, the Amazon DynamoDB may be a database to store user information of all the users. Further, the Amazon S3 may be used as a storage service for storing files, codes, etc.

In some embodiments, the present invention may include Exclusive Surgeries Solutions software platform. Further, the Exclusive Surgeries Solutions software platform includes a login page. Further, an administrator may log in to access a portal of registered users. Further, the registered users may be patients of a medical organization, a medical professional, etc. Subsequently, the administrator may access patient information and medical records of any registered user. Further, the administrator may edit patient information and add a new medical record corresponding to the registered user. Further, the administrator may access a medical record from a medical records list associated with the registered user. Further, the administrator may view information like upload date, as well as have data on payments made and negotiations between office managers and Exclusive Surgeries Network's employees, categories, comments, etc. associated with the medical record. Further, the administrator may download the medical record, share the medical record and add new comments for the medical record.

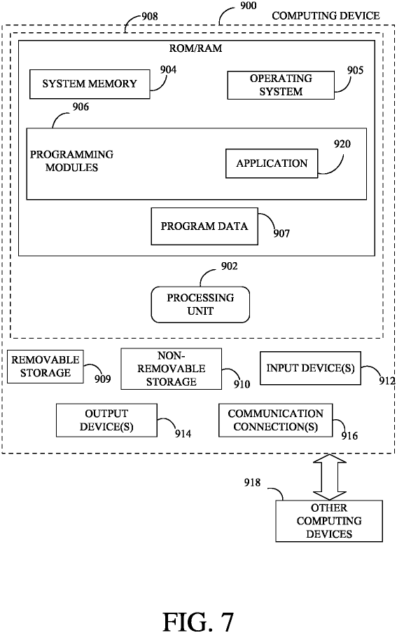

With reference to FIG. 7, a system consistent with an embodiment of the disclosure may include a computing device or cloud service, such as computing device 900. In a basic configuration, computing device 900 may include at least one processing unit 902 and a system memory 904. Depending on the configuration and type of computing device, system memory 904 may comprise, but is not limited to, volatile (e.g., random-access memory (RAM)), non-volatile (e.g., read-only memory (ROM)), flash memory, or any combination. System memory 904 may include operating system 905, one or more programming modules 906, and may include a program data 907. Operating system 905, for example, may be suitable for controlling computing device 900's operation. In one embodiment, programming modules 906 may include image-processing module, machine learning module and/or image classifying module. Furthermore, embodiments of the disclosure may be practiced in conjunction with a graphics library, other operating systems, or any other application program and is not limited to any particular application or system. This basic configuration is illustrated in FIG. 7 by those components within a dashed line 908.

Computing device 900 may have additional features or functionality. For example, computing device 900 may also include additional data storage devices (removable and/or non-removable) such as, for example, magnetic disks, optical disks, or tape. Such additional storage is illustrated in FIG. 7 by a removable storage 909 and a non-removable storage 910. Computer storage media may include volatile and nonvolatile, removable and non-removable media implemented in any method or technology for storage of information, such as computer-readable instructions, data structures, program modules, or other data. System memory 904, removable storage 909, and non-removable storage 910 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information, and which can be accessed by computing device 900. Any such computer storage media may be part of device 900. Computing device 900 may also have input device(s) 912 such as a keyboard, a mouse, a pen, a sound input device, a touch input device, a location sensor, a camera, a biometric sensor, etc. Output device(s) 914 such as a display, speakers, a printer, etc. may also be included. The aforementioned devices are examples and others may be used.

Computing device 900 may also contain a communication connection 916 that may allow device 900 to communicate with other computing devices 918, such as over a network in a distributed computing environment, for example, an intranet or the Internet. Communication connection 916 is one example of communication media. Communication media may typically be embodied by computer-readable instructions, data structures, program modules, or other data in a modulated data signal, such as a carrier wave or other transport mechanism, and includes any information delivery media. The term "modulated data signal" may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 904, including operating system 905. While executing on processing unit 902, programming modules 906 (e.g., application 920 such as a media player) may perform processes including, for example, one or more stages of methods, algorithms, systems, applications, servers, databases as described above. The aforementioned process is an example, and processing unit 902 may perform other processes. Other programming modules that may be used in accordance with embodiments of the present disclosure may include sound encoding/decoding applications, machine learning application, acoustic classifiers, etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid-state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the invention has been explained in relation to its preferred embodiment, it is to be understood that many other possible modifications and variations can be made without departing from the spirit and scope of the invention.

What is claimed is:

1. A method comprising:
receiving, using a communication device, medical data from a user device;
analyzing, using a processing device, the medical data;
receiving, using the communication device, a categorizing keyword from the user device;
analyzing, using the processing device, the categorizing keyword to identify a category type;
organizing, using the processing device, the medical records based on the category type;
storing, using a storage device, the medical data in a database;
transmitting, using the communication device, organized medical records to a medical expert's device;
receiving, using the communication device, expert data from the medical expert's device; and
storing, using the storage device, the expert data with the medical data in the database.

2. The method as claimed in claim 1, wherein the medical data includes a name, age, gender, height, and weight.

3. The method as claimed in claim 1, wherein the medical records includes vaccination records, surgery records, prescriptions, medications, medical conditions, medical test results, and diagnosis results.

4. The method as claimed in claim 1, wherein the category type includes a treatment type, a surgery type, a prescription type, a treatment period type, a medical condition type, a medical organization type, and a medical professional type.

5. The method as claimed in claim 1, wherein the medical data is in an image, text, document, PDF, JPG, JPEG, DICOM, spreadsheet, video, and audio format.

6. The method as claimed in claim 1, wherein the medical data includes a date, time, location, venue, examiner, medical organization, medical professional associated with the treatment type.

7. The method as claimed in claim 1, wherein the expert data includes recommendations, prescriptions, treatment, and diet plans.

8. The method as claimed in claim 1, further comprising:
receiving, using a communication device, medical data from a user device;
analyzing, using a processing device, the medical data;
receiving, using the communication device, a categorizing keyword from the user device;
analyzing, using the processing device, the categorizing keyword to identify a category type;
organizing, using the processing device, the medical records based on the category type;
storing, using a storage device, the medical data in a database;
transmitting, using the communication device, organized medical records to a medical expert's device;
receiving, using the communication device, expert data from the medical expert's device;
storing, using the storage device, the expert data with the medical data in the database;
receiving, using a communication device, a pairing key from at least one medical device;
analyzing, using a processing device, the pairing key based on the receiving;
receiving, using the communication device, device data from the at least one medical device;
analyzing, using the processing device, the device data based on the receiving;
generating, using the processing device, a health report based on analyzing the device data; and
storing, using a storage device, the health report in a database based on the generating.

9. The method as claimed in claim 8, wherein the at least one medical device includes a Blood Pressure monitor, Blood Glucose monitor, ECG machine, EEG machine, pacemakers, and activity trackers.

10. The method as claimed in claim 8, wherein the at least one medical device includes a Bluetooth module and a Wi-Fi module.

11. A method comprising:
receiving, using a communication device, medical data from a user device;
analyzing, using a processing device, the medical data;
receiving, using the communication device, a categorizing keyword from the user device;
analyzing, using the processing device, the categorizing keyword to identify a category type;
organizing, using the processing device, the medical records based on the category type;
storing, using a storage device, the medical data in a database;
transmitting, using the communication device, organized medical records to a medical expert's device;
receiving, using the communication device, expert data from the medical expert's device;
storing, using the storage device, the expert data with the medical data in the database;
receiving, using a communication device, a schedule request from a user device;
analyzing, using a processing device, the schedule request to identify medical data associated with the user;
retrieving, using a storage device, the medical data based on the analyzing;
determining, using the processing device, a treatment type using the medical data;
retrieving, using the storage device, at least one appointment preset associated with the treatment type;
transmitting, using the communication device, the at least one appointment preset to the user device;
receiving, using the communication device, an appointment preset selection from the at least one appointment preset from the user device;
generating, using the processing device, at least one schedule reminder based on the appointment preset selection; and
transmitting, using the communication device, an alert based on the at least one schedule reminder.

12. The method as claimed in claim 11, further comprising:
retrieving, using a storage device, the expert data and the medical data associated with the user;
analyzing, using a processing device, the expert data and the medical data;
determining, using the processing device, susceptibility of illnesses based on the medical data;
determining, using the processing device, treatment of illnesses on the expert data;
generating, using the processing device, a safety report; and
transmitting, using a communication device, the safety report to a user device associated with the user.

13. A non-transitory computer readable medium containing computer-readable instructions stored therein for causing a computer processor to perform operations to:
receive, using a communication device, medical data from a user device;
analyze, using a processing device, the medical data;
receive, using the communication device, a categorizing keyword from the user device;
analyze, using the processing device, the categorizing keyword to identify a category type;
organize, using the processing device, the medical records based on the category type;
store, using a storage device, the medical data in a database;
transmit, using the communication device, organized medical records to a medical expert's device;
receive, using the communication device, expert data from the medical expert's device;
store, using the storage device, the expert data with the medical data in the database;
receive, using a communication device, a pairing key from at least one medical device;
analyze, using a processing device, the pairing key based on the receiving;
receive, using the communication device, device data from the at least one medical device;
analyze, using the processing device, the device data based on the receiving;
generate, using the processing device, a health report based on analyzing the device data; and
store, using a storage device, the health report in a database based on the generating;
receive, using a communication device, a schedule request from a user device;
analyze, using a processing device, the schedule request to identify medical data associated with the user;
retrieve, using a storage device, the medical data based on the analyzing;
determine, using the processing device, a treatment type using the medical data;
retrieve, using the storage device, at least one appointment preset associated with the treatment type;
transmit, using the communication device, the at least one appointment preset to the user device;
receive, using the communication device, an appointment preset selection from the at least one appointment preset from the user device;
generate, using the processing device, at least one schedule reminder based on the appointment preset selection; and
transmit, using the communication device, an alert based on the at least one schedule reminder.
retrieving, using a storage device, the expert data and the medical data associated with the user;
analyzing, using a processing device, the expert data and the medical data;
determining, using the processing device, susceptibility of illnesses based on the medical data;
determining, using the processing device, treatment of illnesses on the expert data;
generating, using the processing device, a safety report; and
transmitting, using a communication device, the safety report to a user device associated with the user.

14. The non-transitory computer readable medium as claimed in claim 13, wherein the medical data includes a name, age, gender, height, and weight.

15. The non-transitory computer readable medium as claimed in claim 13, wherein the medical records includes vaccination records, surgery records, prescriptions, medications, medical conditions, medical test results, and diagnosis results.

16. The non-transitory computer readable medium as claimed in claim 13, wherein the category type includes a treatment type, a surgery type, a prescription type, a treatment period type, a medical condition type, a medical organization type, and a medical professional type.

17. The non-transitory computer readable medium as claimed in claim 13, wherein the medical data is in an image, text, video, and audio format.

18. The non-transitory computer readable medium as claimed in claim 13, wherein the medical data includes a date, time, location, venue, examiner, medical organization, medical professional associated with the treatment type.

19. The non-transitory computer readable medium as claimed in claim 13, wherein the expert data includes recommendations, prescriptions, treatment, and diet plans.

20. The non-transitory computer readable medium of claim 13, wherein the computer-readable instructions stored therein for causing the computer processor to perform operations further to:
retrieve, using a storage device, the expert data and the medical data associated with the user;
analyze, using a processing device, the expert data and the medical data;
determine, using the processing device, susceptibility of illnesses based on the medical data;
determine, using the processing device, treatment of illnesses on the expert data;
generate, using the processing device, a safety report; and
transmit, using a communication device, the safety report to a user device associated with the user.
